(19) United States
(12) Patent Application Publication
(10) Pub. No.: US 2007/0156066 A1
(43) Pub. Date: Jul. 5, 2007

(54) Device for determining the shape of an anatomic surface

(75) Inventors: Shawn E. McGinley, Fort Wayne, IN (US); James E. Grimm, Winona Lake, IN (US); Jody L. Claypool, Columbia City, IN (US)

Correspondence Address: John F. Hoffman, Esq.;BAKER & DANIELS LLP, Suite 800, 111 East Wayne Street, Fort Wayne, IN, 46802, US

(73) Assignee: Zimmer Technology, Inc.

(21) Appl. No.: 11/324,624

(22) Filed: Jan. 3, 2006

(51) Int. Cl. A61B 5/103

(52) U.S. Cl. 600/587

(57) ABSTRACT

The present invention provides a device for determining the shape and/or position of an anatomic surface and converting the data into machine readable form. The device includes a plurality of sensing probes positionable against an anatomic surface and a mechanism able to read the probe positions to determine the shape of the surface. The device may also include a tracking element trackable by a surgical navigation system to determine the position of the probes relative to a surgical coordinate system.

### FIELD OF THE INVENTION

The invention relates to a device for determining the shape of an anatomic feature. In particular this invention relates to a device for determining the shape and/or position of an anatomic surface and converting the data into machine readable form.

### BACKGROUND

Various surgical procedures are aided by knowledge of the shape and location of an anatomic feature. By understanding the shape and/or location of the feature, the surgeon can appropriately treat defects, fashion replacements, position surgical components, and otherwise make surgical decisions relative to a surgical site. Surgical components may include implants, trial implants, drills, burrs, saws, lasers, thermal ablators, electrical ablators, retractors, clamps, cameras, microscopes, guides, and other surgical components. Surgical sites may include a hip joint, knee joint, vertebral joint, shoulder joint, elbow joint, ankle joint, digital joint of the hand or foot, jaw, fracture site, tumor site, and other suitable surgical sites for which shape and location information is desirable.

For example, to fill a lesion at a surgical site, knowledge of the shape of the lesion and surrounding tissue may guide the surgeon in treating the lesion. For example, knowing the shape and location of arthritic lesions on an articular surface of a skeletal joint may aid in determining how to treat the lesions and guide surgical components to the lesions during surgery. Knowledge of the shape and location of joint lesions may also aid in determining whether the lesions can be treated discretely or whether the entire articular surface needs to be replaced.

Knowledge of the shape of a surgical site may aid in forming or choosing a prosthetic replacement for implantation at the surgical site. For example, knowledge of the shape of an articulating surface of a skeletal joint can be used to determine the appropriate size, shape, style, and/or other parameter for a prosthetic replacement. For example, if it is desired to accurately replace a portion of a joint surface to its preoperative shape and position, knowledge of the preoperative shape and position for the particular patient is required. This information can be used to shape an implant or it can be used to choose an implant from a catalog of existing implants and to position the implant to best reproduce the pre-surgical anatomy and finction or to correct a measured pre-surgical deformity.

Knowledge of the shape and location of a surgical site may aid in accurately positioning surgical components at a particular location and in a particular orientation. For example, by knowing where a defect or surgical landmark is located a surgical component can be positioned and oriented relative to the defect or landmark. For example, a surgical component can be positioned at a particular point on a surface normal to the surface, tangent to the surface, or at any other predetermined angle relative to the surface at the point. For example, a cutting instrument could be positioned at a particular location located normal to the surface of the tissue to be cut.

Surgeons typically gain knowledge of the shape and location of surgical sites preoperatively by using imaging technologies such as x-ray filming, fluoroscopy, computer aided tomography (CAT) scanning, and magnetic resonance imaging (MRI) scanning. These methods are limited. For example, x-ray filming only provides two-dimensional profile information and only for dense, radiopaque features. CAT scans are essentially a series of x-ray films taken in rotation about an object and computerized to provide three-dimensional information. They are also limited by the nature of the x-ray penetration and only work well for dense, radiopaque features. In addition, the x-ray technician or computer software must determine what recorded x-ray intensity corresponds to the actual surface of an anatomic feature and the value chosen can give varying results for the actual shape of the feature. MRI scans are similar to CAT scans in that they are three-dimensional representations made up of a series of two-dimensional scans through an object. The scans are made by exposing the object to high magnetic fields to determine the atomic makeup of the object being scanned. MRI scans have various limitations including the inability to be used around metallic objects such as previously implanted prostheses. Finally, these preoperative techniques are time consuming, relatively expensive, and cannot account for changes that occur in the anatomy between the time the image is produced and the time of surgery.

Surgeons gain knowledge of the shape and location of surgical sites intraoperatively by using palpation, direct observation, and direct measurement using rulers, calipers, and angle gauges. These techniques are limited in that they are time consuming, relatively inaccurate, and can only practically provide measurement of a relatively few points at the surgical site. Manually measuring enough points to accurately represent an anatomic surface would take far too long to be practical.

Many surgical procedures are now performed with surgical navigation systems in which sensors detect tracking elements attached in known relationship to an object in the surgical suite such as a surgical instrument, implant, or patient body part. The sensor information is fed to a computer that then triangulates the position of the tracking elements within the surgical navigation system coordinate system. Thus the computer can resolve the position and orientation of the object and display the position and orientation for surgeon guidance. By digitizing patient image data and relating it to the surgical navigation system coordinate system, the position and orientation of an object can be shown superimposed on an image of the patient's anatomy obtained via x-ray, CAT scan, MRI scan, or other imaging technology.

### SUMMARY

The present invention provides an apparatus for determining the shape and/or position of an anatomic surface and converting the data into machine readable form.

In one aspect of the invention, an apparatus for determining the shape of an anatomic surface, includes a base and a plurality of probes mounted for translation relative to the base. The probes are simultaneously positionable in contact with the anatomic surface. The apparatus further includes means for converting the individual probe positions into machine readable form.

In another aspect of the invention, a method for determining the shape of an anatomic surface includes simultaneously contacting a plurality of probes to an anatomic surface; and converting the probe positions into machine readable form.

### BRIEF DESCRIPTION OF THE DRAWINGS

Various examples of the present invention will be discussed with reference to the appended drawings. These drawings depict only illustrative examples of the invention and are not to be considered limiting of its scope.

FIG. 1 is a side elevation view of an illustrative surface contour reader according to the present invention;

FIG. 2 is a front elevation view of the illustrative surface contour reader of FIG. 1;

FIG. 3 is a side sectional view of the illustrative surface contour reader of FIG. 1;

FIGS. 4 and 5 are side views of alternative sensing pin arrangements for the illustrative surface contour reader of FIG. 1;

FIG. 6 is a schematic view of a computer and display used to process and display information obtained with the contour reader of FIG. 1; and

FIG. 7 is a side elevation view of the illustrative surface contour reader of FIG. 1 in use to determine the shape of a portion of femur.

### DESCRIPTION OF THE ILLUSTRATIVE EXAMPLES

Embodiments of a device for determining the shape of an anatomic surface include a plurality of probes mounted for translation relative to a datum plane for simultaneously determining the three-dimensional coordinate positions of a plurality of points on the anatomic surface and converting the coordinate positions into machine readable form. For example, the datum plane may define a two dimensional datum coordinate system. The probes may have a first, or initial, position relative to the datum plane. The probes may be simultaneously positionable in contact with the anatomic surface such that for any given relative positioning of the device and the anatomic surface, each probe will translate to a second position relative to the datum plane depending on the shape and orientation of the surface and the orientation of the device. The second position of each probe defines a third dimension relating the point where the probe contacts the anatomic surface to the two dimensional coordinate system defined by the datum plane. Thus, by knowing the location of each probe within the two dimensional datum coordinate system and the second position of the probe, a sample of points on the surface may be determined in three dimensions.

The probes may take a variety of forms including buttons, rods, tubes, pins, wires, and/or other suitable forms. For example the probes may be in the form of axially elongated cylindrical pins mounted for axial translation relative to the datum plane.

The probes may be arranged as a regular array within the datum plane. For example the probes may be arranged in a rectangular grid of x columns by y rows. Alternatively, the probes may be arranged in concentric rings of probes or in any other desirable pattern. The predetermined position of each probe within the datum plane may be recorded as a Cartesian x-axis/y-axis ordered pair, as a polar radius/angle ordered pair, and/or by any other suitable position recordation system. The position of the anatomic surface contacting portion of each probe may be recorded as a z-axis distance spaced from the datum plane. The datum plane may be defined by a solid mounting surface attached to a device base. The mounting surface may include a plurality of through holes in which the probes translate normal to the surface. The probes may form a close slip fit within the holes to minimize side to side motion of the probes. The probes may be biased into the first position in which a portion of each probe is in contact with the datum plane and the surface contacting end of each probe is a predetermined distance from the datum plane.

The device includes a mechanism for determining the probe position relative to the datum plane. This position may be measured directly or a translation distance may be measured and compared to a known initial position to determine the current probe position. The mechanism for determining the probe position may generate an electrical signal relatable to the probe position and/or displacement and transmit the signal to a computer for recording the position of each probe. The mechanism for determining the probe position may include an emitter, a detector, and a timer. For example an emitter may emit an electromagnetic wave such as light toward one end of the probe. The wave may reflect from the end of the probe and be detected by a detector. The time for the wave to pass from the emitter to the detector may be measured and converted into a probe position. In another example, the mechanism for determining the probe position may include an emitter and detector directed toward a side of the probe containing contrasting indicia such as black and white markings. As the probe translates, the indicia move past the emitter and detector creating electrical pulses. The computer can count the pulses and convert the number of pulses into a translation distance based on the known spacing of the indicia. The position of the probe can be determined by comparing the translation distance to a known initial position. In another example, the mechanism for determining the probe may include an electromagnetic coil surrounding a portion of each probe such that movement of the probe within the coil changes the inductance of the coil. A current through the coil can then be related to the probe position. In another example, the mechanism for determining the probe position may include a linear potentiometer in which changing probe position changes a conductive path length within the potentiometer to change the resistance of the potentiometer. A voltage measured across the potentiometer will be proportional to the probe position and can be used to determine the probe position. The mechanism for determining the probe position may include other mechanisms including proximity transducers, ultrasonic distance measuring arrangements, Hall Effect transducers, and/or any suitable mechanism.

The device may include a computer and software for converting the measured coordinates into a computer model of the anatomic surface. The computer model may be a simple point cloud of all of the measured points. The computer model may include interpolated points between the measured points to provide a smoother model. The computer model may include polygons or other surface models fit to the point data by the computer.

The device for determining the shape of an anatomic surface may be used to make single instantaneous measurements. For example, the device may be positioned with the probes contacting a surface and then a signal may be given to a computer to read the probe positions such as by pressing a button. If additional readings are desired, the device may be repositioned and the button pressed again. Each press of the button will yield a set of coordinates corresponding to a single reading for each probe position. Alternatively, the computer may automatically record a set of coordinates at predetermined time intervals. The frequency with which the computer records the coordinates may be called a frame rate. For example, the computer may record a set of coordinates several times each second. In this case, the device may be passed over an anatomic surface continuously while the computer automatically records the data. The faster the frame rate, or the more times per second that the computer records probe positions, the smoother the resulting surface model will be. Whether the computer is triggered manually to record each data set or automatically, the computer can compare the individual sets and piece them together to form a single model of the anatomic surface.

The device may include one or more tracking elements detectable by a surgical navigation system such that the three dimensional position of the tracking elements can be related to a surgical navigation coordinate system. For example, a surgical navigation system may include multiple sensors at known locations that feed tracking element position information to a computer. The computer may then use the position information from the multiple sensors to triangulate the position of each tracking element within the surgical navigation coordinate system. The surgical navigation system can then determine the position and orientation of the probes within the surgical navigation coordinate system by detecting the position and orientation of the tracking elements and then resolving the position and orientation of the probes from the known relationship between the tracking elements and the probes. Tracking elements may be detectable by imaging, acoustically, electromagnetically, and/or by other suitable detection means. Furthermore, tracking elements may be active or passive. Examples of active tracking elements may include light emitting diodes in an imaging system, ultrasonic emitters in an acoustic system, and electromagnetic field emitters in an electromagnetic system. Examples of passive tracking elements may include elements with reflective surfaces.

The device of the present invention may be used in a variety of ways. It may be used to measure the shape of an anatomic surface. The shape information may then be used to produce a computer model. The information may be used to detect defects in the surface measured. For example, if a healthy example of the measured surface is smooth, the measurements may be used to identify defects such as lesions, pits, cracks, and/or other defects. The size, shape, and position of the defects may be determined by the computer model to help in treating the discrete defects or to help in making a determination that the entire surface needs to be replaced. The information may be used to model the shape of a surface to be replaced. For example the information may be used to select the size and shape of a replacement implant from a catalog of pre-existing prostheses. The information may be used to identify landmarks on the surface such as condyles, epicondyles, trochanters, fossa, foramen, sulci, and/or other landmarks. For example, the computer software may include algorithms for analyzing the surface data and comparing it to a catalog of standard anatomic relationships to identify the presence of a particular landmark. The information may be used to guide the placement of surgical components intraoperatively. The information may be presented to the user as a graphical image on a computer display, as alphanumeric information, as audible commands or tones, and/or by other suitable presentation methods.

In another example, a landmark or other feature of the surface measured with the device may be matched to a surface in a computer model created from x-ray films, CAT scans, MRI scans, and/or other measuring methods. For example, a detailed model of a patient's anatomy may be created from CAT scans prior to surgery. During surgery, a surgical coordinate system may be established. Tracking elements placed on the patient and surgical components in the operating environment permit tracking of the objects within the surgical coordinate system. The device of the present invention may also include a tracking element relating it to the surgical coordinate system. The anatomic model created before surgery can be indexed to the surgical coordinate system by measuring a subset of the modeled anatomy intraoperatively with the present invention. The computer may compare the measured portion to the predetermined model until the measured portion matches a portion of the predetermined model. When a match is found, the computer may translate the predetermined model into the surgical navigation coordinate system so that the predetermined model and the current surgical navigation coordinate system are in registration with one another.

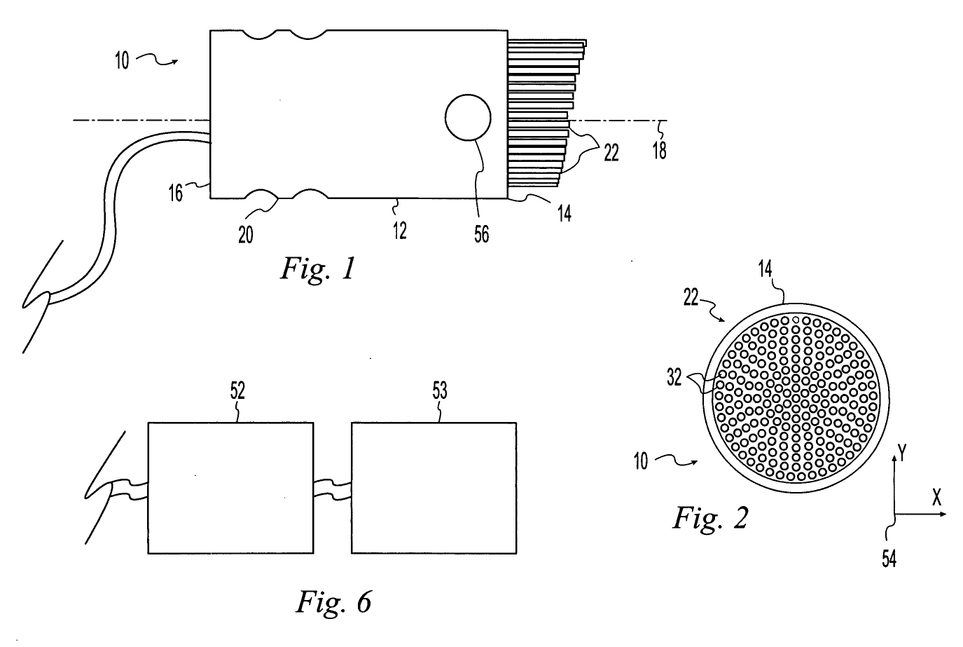

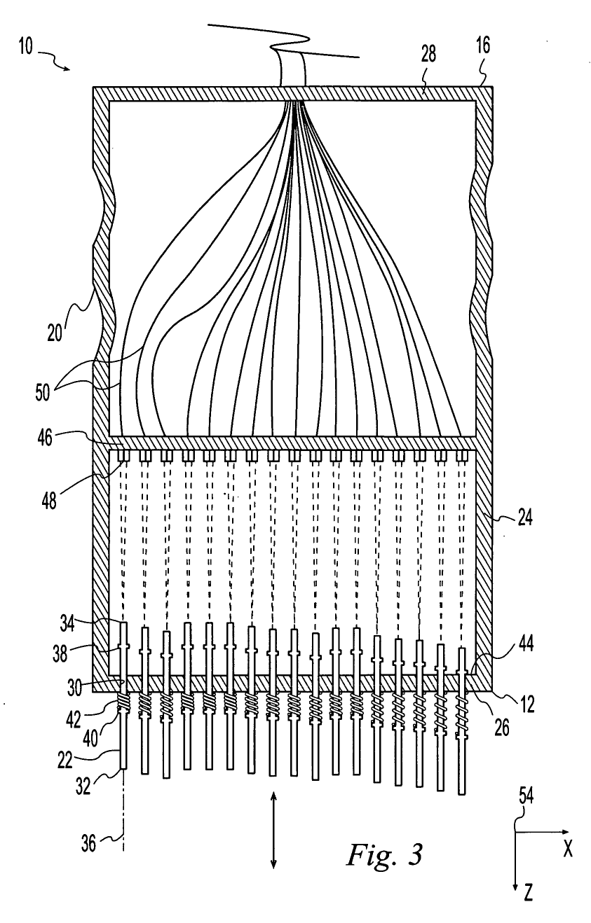

FIGS. 1-3 depict a device for determining the shape of an anatomic surface in the form of a hand-held surface contour reader 10. The reader 10 includes a housing 12 having a proximal end 14, a distal end 16, and an axis 18 extending between the proximal and distal ends 14, 16. The housing 12 includes a handle portion 20 adjacent the distal end 16 and an array of pins 22 extends from the housing 12 at the proximal end 14. The housing 12 is in the form of a hollow cylinder having a side wall 24, a proximal end wall 26, and a distal end wall 28 (FIG. 3). Each pin in the array of pins 22 is mounted in a bore 30 formed through the proximal end wall 26 for axial translation within the bore 30. Each pin 22 includes a proximal end 32, a distal end 34, and an axis 36 extending between the proximal and distal ends 32, 34. Each pin 22 further includes a stop 38 and a spring retainer 40 in the form of annular projections intermediate the proximal and distal ends 32, 34. A coil spring 42 around each pin 22 abuts the proximal end wall 26 and the spring retainer 40 and biases the pin 22 proximally. The proximal end wall 26 of the housing 12 defines an inner datum plane 44 against which each pin stop 38 is biased in a rest state. Pressure on the proximal end 32 of each pin 22 causes it to translate within the bore 30 distally against spring pressure. The pins 22 in FIG. 3 are shown displaced varying amounts as they would be if the proximal ends 32 were contacting an uneven surface.

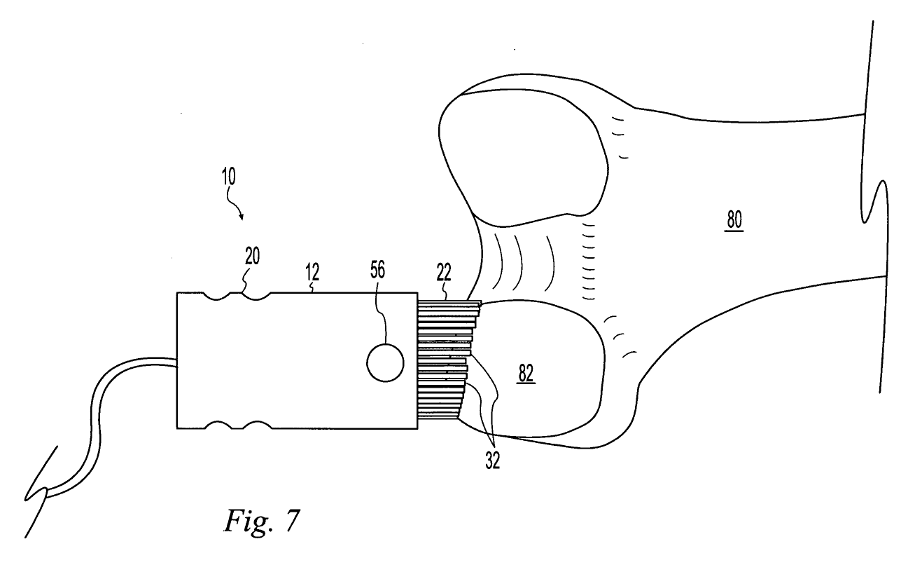

An intermediate wall 46 within the housing supports an array of pin position detectors in the form of emitter/detector pairs 48. Each emitter directs light toward the distal end 34 of a corresponding one of the pins 22. The light is reflected from the distal end 34 of the pin 22 and is detected by a detector. The emitters and detectors are connected via wires 50 to a computer (FIG. 6) 52 including a timing device. The computer triggers the emitter and starts a timer. The computer then records the time the light takes to reach the detector and converts the time into a pin position.

The array of pins 22 is arranged in concentric circles lying in the datum plane 44. The position of each pin 22 within the datum plane 44 is predetermined and fixed and defined relative to a reader coordinate system 54 (FIG. 2) by an (x,y) coordinate pair. The position of the proximal end 32 of each pin 22 is variable depending on the translated position of each pin 22. The position of the proximal end 32 of each pin 22 relative to the datum plane 44 defines a third dimension, or z-dimension (FIG. 3), relative to the datum plane 44. Thus, by knowing the location of each probe within the two dimensional datum coordinate system and the pin translation position, the three dimensional position of the proximal end 32 of each pin 22 is defined relative to the reader coordinate system 54. A tracking element 56 in the form of an electromagnetic coil is mounted to the housing 12. A surgical navigation system is able to detect the position of the coil and resolve the position and orientation of the housing and thus the origin of the reader coordinate system 54. The surgical navigation system can relate the reader coordinate system to the surgical coordinate system so that the position of the proximal end 32 of each pin 22 is known in the surgical coordinate system.

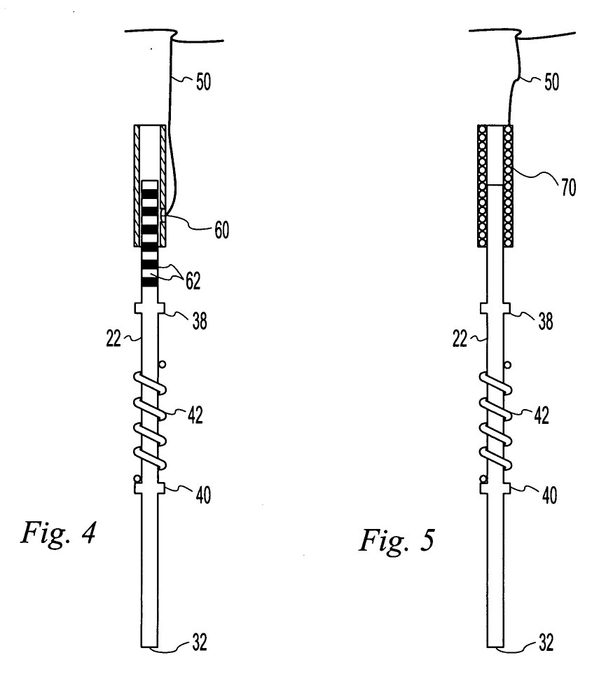

FIG. 4 depicts an alternative arrangement for the pin position detectors in the form of emitter/detector pairs 60 aimed at the side of each pin 22. The distal portion of each pin includes contrasting indicia 62 in the form of alternating light and dark markings. As the pin 22 translates, the indicia move past the emitter and detector creating electrical pulses. The computer 52 can count the pulses and convert the number of pulses into a translation distance based on the known spacing of the indicia 62. The position of the pin 22 can be determined by comparing the translation distance to a known initial position.

FIG. 5 depicts another alternative arrangement for the pin position detectors in the form of an electromagnetic coil 70 surrounding the distal portion of each pin 22 such that movement of the pin 22 within the coil 70 changes the inductance of the coil. A current or voltage through or across the coil 70 can then be related to the pin 22 position. Alternatively, the coil 70 may be setup as a potentiometer in which opposite sides of the coil 70 can be connected to an electrical source and a portion of the inside of the coil may be uninsulated such that as the pin 22 slides within the coil it shorts the opposing sides. The further the pin 22 extends into the coil 70, the lower the overall resistance of the coil. Applying a current to the coil will result in a voltage drop across the coil 70 that is proportional to the pin 22 position and which can be measured to determine the pin 22 position.

The computer 52 records the three-dimensional position of the end 32 of each of the pins 22. The computer 52 can then process the position information to produce a computer model of the shape of the anatomic surface which the ends 32 are contacting. The information may be presented to the user as a graphical image on a computer display 53, as alphanumeric information, as audible commands or tones, and/or by other suitable presentation and/or feedback methods.

For example, FIG. 7 depicts the reader 10 in use to measure the surface of the distal portion of a femur 80. As the ends 32 of the pins 22 are pressed against the surface of the femoral condyle 82, each pin 22 will translate distally into the housing 12 a distance determined by the orientation of the reader 10 relative to the surface and the shape of the surface. The relative translation of the pins 22 defines the shape of the surface. The computer 52 can then produce a model of the surface by recording the z-axis distance of the end 32 of each pin with its predetermined x-axis and y-axis position within the reader coordinate system. This computer model can be used in a variety of ways to aid the surgeon in treating the patient. For example, the model can be displayed graphically to show the surgeon the shape of the condyle. This may be helpful where the surgeon is approaching the surgical site through a small incision that makes direct visualization difficult. The computer 52 can use algorithms to process the model data and identify abrupt changes in the slope of the condyle surface such as might be present around a condylar defect or lesion. The computer can emphasize these abrupt changes, for example, by displaying them in a contrasting color for enhanced viewing by the surgeon. This improved view of the surface of the condyle can help the surgeon decide how to treat the condylar defects such as by abrasion, discrete resurfacing, or total resurfacing. The computer model may also be used to select the size and shape of an appropriate condylar implant from a catalog of pre-existing prostheses. The computer model may also be used to identify landmarks on the surface such as condyles, epicondyles, trochanters, fossa, foramen, sulci, and/or other landmarks. For example, the computer software may include algorithms for analyzing the surface data and comparing it to a catalog of standard anatomic relationships to help identify a protrusion such as an epicondyle and indicate its location to the surgeon.

In order to measure an area larger than the pin array 22, the reader 10 can be repositioned on the condyle and another set of pin positions can be recorded. The computer 52 can include an algorithm that analyzes multiple sets of surface data to identify matching areas and stitch the data sets together into a single model of a larger surface. The computer can be manually triggered to record the pin 22 positions such as by pressing a button when the reader 10 is engaged with a surface to be read. The reader 10 can be repositioned and the computer 52 triggered again to record multiple areas. Alternatively, the computer can automatically record a set of pin positions at predetermined time intervals so that the user can move the reader over a surface while the computer records pin positions to scan a surface larger than the pin array 22. Each set of data is called a frame and the frequency of recording the data is called a frame rate. The faster the frame rate, or the more times per second that the computer records probe positions, the smoother the resulting surface model will be. To generate a model of the surface of the distal femur 80, the user passes the reader 10 over the bone surface while the computer records pin positions several times per second. After the desired area has been scanned, the computer 52 compiles the collected data into a single model of the scanned area discarding redundant data if necessary.

With a surgical navigation system activated to track the tracking element 56, the location of the condylar surface model can be related to the surgical coordinate system. The tracking element 56 position relative to the reader coordinate system is fixed and predetermined. At any given instant, the position of the tracking element 56 within the surgical coordinate system is recorded by the surgical navigation system such that the pin positions measured relative to the reader coordinate system can be transformed into the surgical coordinate system and related to other objects registered in the surgical coordinate system. This use with a surgical navigation system expands the use of the reader 10 so that the computer model includes not only size and shape information pertaining to the condylar surface but also the location within the operating environment. This additional information can be used to guide cutting instruments to intersect the surface in desired orientations and positions, to position implants, and/or other surgical purposes.

In some situations it may be desirable to use a predetermined model of the surgical anatomy such as one generated from CAT scan or MRI scan data. The reader 10 can be used to align the predetermined computer model with the actual position of the patient in the operating environment. The reader is engaged with a portion of the surgical anatomy intraoperatively to generate a model of the portion. This intraoperative model is compared to the predetermined model until the portion read by the reader 10 matches a portion of the predetermined model. The computer then has sufficient information to transform the predetermined model so that it is indexed with the surgical coordinate system. In this example, the reader is used to generate a temporary model for aligning a larger predetermined model at the time of surgery. Once the predetermined model is aligned, the temporary model can be discarded.

The reader 10 can be produced in any desirable size with any desirable size of pin array. For surgery through a small incision, a relatively small pin array may be advantageous. The small array may be manipulated through the small incision to scan or sequentially engage a larger surface. Alternatively, where space permits, a relatively large pin array may be used that can engage and record the shape of a relatively large surface all at once. The resolution of the data collected by the reader 10 can be varied by varying the pin 22 spacing in the datum plane. Relatively large spacing and fewer pins will produce a relatively coarse model while relatively small spacing and more pins will produce a relatively fine model.

Although examples of a device for determining the shape of an anatomic surface and its use have been described and illustrated in detail, it is to be understood that the same is intended by way of illustration and example only and is not to be taken by way of limitation. The invention has been illustrated as a hand held anatomic contour reader in use to determine the shape of a portion of the surface of the distal femur. However, the device may be alternatively configured and may be used to determine the shape of other anatomic surfaces at other locations within a patient's body. Accordingly, variations in and modifications to the device for determining the shape of an anatomic surface and its use will be apparent to those of ordinary skill in the art, and the following claims are intended to cover all such modifications and equivalents.

1. An apparatus for determining the shape of an anatomic surface, the apparatus comprising:
a base;
a plurality of probes mounted for translation relative to the base, the probes being simultaneously positionable in contact with the anatomic surface; and
means for converting the individual probe positions into machine readable form.

2. The apparatus of claim 1 wherein the probes comprise an array of cylindrical pins mounted for translation in holes formed in the base.

3. The apparatus of claim 2 wherein the base includes a datum surface, the holes being formed through the datum surface, and the pins are spring biased into contact with the datum surface.

4. The apparatus of claim 3 wherein the pins each include a stop abuttable with the datum surface and a spring retainer formed opposite the stop, a spring being positioned between the base and the spring retainer to bias each pin stop into abutment with the datum surface.

5. The apparatus of claim 1 wherein the means for converting the individual probe positions into machine readable form comprises a means for generating an electrical signal proportional to the probe position and further wherein the apparatus comprises a computer linked to the means for generating an electrical signal such that the computer can record the position of each probe.

6. The apparatus of claim 5 wherein the computer further comprises means for modeling the shape of the anatomic surface from the probe position locations.

7. The apparatus of claim 6 further comprising means for displaying the model of the anatomic surface for surgeon reference.

8. The apparatus of claim 5 wherein the computer further comprises means for analyzing the probe position locations to identify an anatomic landmark.

9. The apparatus of claim 5 further comprising a tracking element trackable by a surgical navigation system within a surgical coordinate system.

10. The apparatus of claim 9 wherein the computer further comprises means for indexing prior anatomic image data to the surgical coordinate system by matching the probe positions to a shape in the prior data.

11. The apparatus of claim 5 wherein the means for generating an electrical signal proportional to the probe positions comprises a light emitter and a light detector.

12. The apparatus of claim 11 wherein each probe includes a first end for contacting the anatomic surface, a second end, and an axis extending between the first and second ends, each probe being mounted to the base for axial translation, the light emitter being directed toward the second end and the light detector receiving light reflected from the second end.

13. The apparatus of claim 11 wherein each probe includes a first end for contacting the anatomic surface, a second end, an axis extending between the first and second ends, and a longitudinal side surface, each probe being mounted to the base for axial translation, the side surface including alternating contrasting indicia, the emitter and detector being directed toward the indicia.

14. The apparatus of claim 5 wherein each probe includes a first end for contacting the anatomic surface, a second end, and an axis extending between the first and second ends, each probe being mounted to the base for axial translation, the means for generating an electrical signal proportional to the probe positions comprising a linear potentiometer associated with each probe, each probe being linked to its corresponding potentiometer to vary the resistance of the potentiometer as the probe translates.

15. A method for determining the shape of an anatomic surface, the method comprising: simultaneously contacting a plurality of probes to an anatomic surface; and
converting the probe positions into a first set of machine readable data.

16. The method of claim 15 wherein converting the probe positions into machine readable data further comprises generating an electrical signal relatable to the probe position.

17. The method of claim 16 further comprising recording the position of each probe in a computer memory.

18. The method of claim 15 further comprising modeling the shape of the anatomic surface from the probe position locations.

19. The method of claim 15 further comprising identifying a landmark by comparing the probe positions to a catalog of known landmark geometries.

20. The method of claim 15 further comprising tracking the probes within a surgical coordinate system.

21. The method of claim 20 further comprising:
comparing the probe positions to a previously generated anatomic model to find a match between a portion of the intraoperative probe positions and a portion of the anatomic model; and
transforming the previously generated anatomic model into the surgical coordinate system to index the previously generated anatomic model to the current surgical environment.

22. The method of claim 15 further comprising:
repositioning the probes on the anatomic surface;
converting the new probe positions into a second set of machine readable data; and
combining the first and second sets of data into a single model of the anatomic surface.

23. The method of claim 15 further comprising periodically recording the pin positions while scanning the probes over the anatomic surface; and combining the periodically recorded probe positions into a single model of the anatomic data.

24. The method of claim 15 further comprising: analyzing the data to identify defects in the anatomic surface.

25. An apparatus for determining the shape of an anatomic surface, the apparatus comprising:
a base;
a plurality of probes mounted for translation relative to the base, the probes being simultaneously positionable in contact with the anatomic surface; and
at least one sensor associated with the probes, the at least one sensor operable to detect the position of each of the plurality of probes.

26. The apparatus of claim 25, further comprising conversion means for converting the individual probe positions into machine readable form, the conversion means comprising generating means for generating an electrical signal proportional to the probe position and a computer linked to the generating means such that the computer can record the position of each probe.

27. The apparatus of claim 26, wherein the computer further comprises modeling means for modeling the shape of the anatomic surface from the probe position locations.

28. The apparatus of claim 26, wherein the computer further comprises analyzing means for analyzing the probe position locations to identify an anatomic landmark.

29. The apparatus of claim 25, further comprising a tracking element trackable by a surgical navigation system within a surgical coordinate system.

30. The apparatus of claim 25, further comprising a computer and a tracking element trackable by a surgical navigation system within a surgical coordinate system, wherein the computer comprises indexing means for indexing prior anatomic image data to the surgical coordinate system by matching the probe positions to a shape in the prior data.

31. The apparatus of claim 25, wherein the at least one sensor comprises a light emitter and a light detector.

32. The apparatus of claim 25, wherein each probe includes a first end for contacting the anatomic surface, a second end, and an axis extending between the first and second ends, each probe being mounted to the base for axial translation, the at least one sensor being directed toward the second end and the at least one sensor receiving light reflected from the second end.

33. The apparatus of claim 25, wherein each probe includes a first end for contacting the anatomic surface, a second end, an axis extending between the first and second ends, and a longitudinal side surface, each probe being mounted to the base for axial translation, the side surface including alternating contrasting indicia, the at least one sensor being directed toward the indicia.

34. The apparatus of claim 25, wherein each probe includes a first end for contacting the anatomic surface, a second end, and an axis extending between the first and second ends, each probe being mounted to the base for axial translation, the at least one sensor comprising a linear potentiometer associated with each probe, each probe being linked to its corresponding potentiometer to vary the resistance of the potentiometer as the probe translates.
